(19) United States
(12) Patent Application Publication
(10) Pub. No.: US 2024/0315619 A1
(43) Pub. Date: Sep. 26, 2024

(54) Blood Collection Kit With Blood Collection Devices of Multiple Sizes and Associated Sizing Systems and Methods

(71) Applicant: Becton, Dickinson and Company, Franklin Lakes, NJ (US)

(72) Inventors: Scott Wentzell, Suffern, NY (US); Charles Peter Althoff, West Nyack, NY (US); Anthony V. Torris, Montclair, NJ (US); Vlad Yakhnich, Park Ridge, NJ (US)

(21) Appl. No.: 18/574,841

(22) PCT Filed: Jun. 23, 2022
(86) PCT No.: PCT/US22/34621

§ 371 Date: Dec. 28, 2023

Related U.S. Application Data
(60) Provisional application No. 63/216,230, filed on Jun. 29, 2021.

(51) Int. Cl. A61B 5/15; A61B 5/00

(52) U.S. Cl. CPC A61B 5/150068; A61B 5/150022; A61B 5/742

(57) ABSTRACT

A kit of parts for obtaining a capillary blood sample includes a plurality of capillary blood collection devices of different sizes and a sizing tool for identifying which of the plurality of blood collection devices to use for a particular patient's finger. Each blood collection device of the plurality of blood collection devices can include a finger holder with a finger receiving portion designed for a unique ideal finger size and configured for use with fingers within a unique size range. Further, the plurality of devices are configured such that a majority of fingers for a population of patients are within one of the unique size ranges for the plurality of capillary blood collection devices.

### CROSS-REFERENCE TO RELATED APPLICATION

The present application claims priority to U.S. Provisional Application Ser. No. 63/216,230, filed Jun. 29, 2021, entitled “Blood Collection Kit with Blood Collection Devices of Multiple Sizes and Associated Sizing Systems and Methods”, the entire disclosure of which is hereby incorporated by reference in its' entirety.

### BACKGROUND OF THE INVENTION

### Field of the Invention

The present disclosure relates generally to devices for obtaining a biological sample. More particularly, the present disclosure relates to finger-based capillary blood collection devices, which can be provided as a set or kit including multiple devices of different sizes optimized to fit most individuals in a population of possible patients. The present disclosure is also directed to systems and methods for determining correct sizing for such blood collection devices.

### Description of Related Art

Devices for obtaining and collecting biological samples, such as blood samples, are commonly used in the medical industry. One type of blood collection that is commonly done in the medical field is capillary blood collection, which is often done to collect blood samples for testing. Certain diseases, such as diabetes, require that a patient's blood be tested on a regular basis to monitor, for example, the patient's blood sugar levels. Additionally, test kits, such as cholesterol test kits, often require a blood sample for analysis. The blood collection procedure usually involves pricking a finger or other suitable body part in order to obtain the blood sample. Typically, the amount of blood needed for such tests is relatively small and a small puncture wound or incision normally provides a sufficient amount of blood for these tests. Various types of lancet devices have been developed, which are used for puncturing the skin of a patient to obtain a capillary blood sample from the patient.

Many different types of lancet devices are commercially available to hospitals, clinics, doctors' offices, and the like, as well as to individual consumers. Such devices typically include a sharp-pointed member, such as a needle, or a sharp-edged member, such as a blade, that is used to make a quick puncture wound or incision in the patient's skin in order to provide a small outflow of blood. In order to simplify capillary blood collection, lancet devices have evolved into automatic devices that puncture or cut the skin of the patient upon actuation of a triggering mechanism. In some devices, the needle or blade is kept in a standby position until it is triggered by the user. Upon triggering, the needle or blade punctures or cuts the skin of the patient, for example, on the finger. Often, a spring is incorporated into the device to provide the “automatic” force necessary to puncture or cut the skin of the patient.

One type of contact activated lancet device that features automatic ejection and retraction of the puncturing or cutting element from and into the device is U.S. Pat. No. 9,380,975, which is owned by Becton, Dickinson and Company, the assignee of the present application. This lancet device includes a housing and a lancet structure having a puncturing element. The lancet structure is disposed within the housing and adapted for movement between a retaining or pre-actuated position wherein the puncturing element is retained within the housing, and a puncturing position, wherein the puncturing element extends through a forward end of the housing. The lancet device includes a drive spring disposed within the housing for biasing the lancet structure toward the puncturing position, and a retaining hub retaining the lancet structure in the retracted position against the bias of the drive spring. The retaining hub includes a pivotal lever in interference engagement with the lancet structure. An actuator within the housing pivots the lever, thereby moving the lancet structure toward the rearward end of the housing to at least partially compress the drive spring, and releasing the lever from interference engagement with the lancet structure. The blood sample that is received is then collected and/or tested. This testing can be done by a Point-of-Care (POC) testing device or it can be collected and sent to a testing facility.

Use of lancet devices for capillary blood collection can be complex requiring a high skill level for the healthcare worker performing the blood collection procedure. The multi-step nature of the capillary blood collection process can introduce several variables that may cause sample quality issues, such as hemolysis, inadequate sample stabilization, and micro-clots. The use of lancet devices for obtaining blood samples can result in several variables that effect the collection of the capillary blood sample, including, but not limited to, holding the lancet still during the testing, obtaining sufficient blood flow from the puncture site, adequately collecting the blood, preventing clotting, and the like. Some of the most common sources of process variability are: (1) inadequate lancing site cleaning and first drop removal which can potentially result in a contaminated sample; (2) inconsistent lancing location and depth which could potentially result in insufficient sample volume and a large fraction of interstitial fluid; (3) inconsistent squeezing technique and excessive pressure near the lancing site to promote blood extraction (e.g., blood milking) which could potentially result in a hemolyzed sample; (4) variable transfer interfaces and collection technique which could potentially result in a hemolyzed or contaminated sample; and (5) inadequate sample mixing with an anticoagulant which could potentially result in micro-clots.

While capillary blood collection devices and assemblies have been developed to simplify the capillary blood collection processes, such as finger-based capillary blood collection devices configured to lance and squeeze a finger, collect, stabilize, and dispense a blood sample in a controlled manner, problems related to blood sample quality can still occur. Attempting to perform a blood draw procedure using a device that is not correctly sized for a patient's finger can exacerbate such blood sample quality problems. For example, an incorrectly sized blood collection device may apply pressure to the patient's finger in an imprecise or inconsistent manner, resulting in poor blood flow from the lanced finger. Also, use of a blood collection device that is not correctly sized for a patient's finger can increase patient discomfort. The blood collection devices and sizing systems and methods of the present disclosure are configured to improve sizing for blood collection devices to improve blood sample quality.

### SUMMARY OF THE INVENTION

According to an aspect of the present disclosure, a kit of parts for obtaining a capillary blood sample includes a plurality of capillary blood collection devices of different sizes and a sizing tool for identifying which of the plurality of blood collection devices to use for a particular patient's finger. Each blood collection device of the plurality of blood collection devices can include a finger holder with a finger receiving portion designed for a unique ideal finger size and configured for use with fingers within a unique size range. Further, the plurality of devices are configured such that a majority of fingers for a population of patients are within one of the unique size ranges for the plurality of capillary blood collection devices.

According to another aspect of the disclosure, a method of making a plurality of blood collection devices sized for use with at least a majority of patient finger sizes for a patient population includes steps of: determining unique size ranges for each of the plurality of blood collection devices; determining a unique ideal size for each device of the plurality of blood collection devices, wherein the unique ideal size for each device is within the determined size range for the respective device of the plurality of blood collection devices; and making the plurality of blood collection devices with dimensions corresponding to the determined unique ideal size for each of the plurality of blood collection devices.

According to another aspect of the disclosure, a computer-implemented method for determining a correct blood collection device size for a patient's finger includes receiving, with at least one computer processor, at least one image of the patient's finger; and processing, with the at least one computer processor, the received at least one image to determine at least one dimension of the patient's finger. The method further includes causing, with the at least one computer processor, an output device to provide a visual indication of which blood collection device of a plurality of blood collection devices having unique finger size ranges to use for the patient's finger determined based, at least in part, on the determined at least one dimension of the patient's finger and the unique size ranges for the plurality of blood collection devices.

Non-limiting illustrative examples of embodiments of the present disclosure will now be described in the following numbered clauses:

Clause 1: A kit of parts for obtaining a capillary blood sample, the kit comprising: a plurality of capillary blood collection devices of different sizes; and a sizing tool for identifying which of the plurality of blood collection devices to use for a particular patient's finger, wherein each blood collection device of the plurality of blood collection devices comprises a finger holder comprising a finger receiving portion designed for a unique ideal finger size and configured for use with fingers within a unique size range, and wherein the unique size ranges for the plurality of blood collection device are selected such that a majority of fingers for a population of patients are within one of the unique size ranges for the plurality of capillary blood collection devices.

Clause 2: The kit of clause 1, wherein the ideal finger sizes comprise an ideal finger width, ideal finger height, and/or ideal finger length, and wherein the unique size ranges comprise a range of finger widths, a range of finger heights, and/or a range of finger lengths.

Clause 3: The kit of clause 1 or clause 2, wherein the finger holders of the blood collection devices further comprise an actuation portion and a port.

Clause 4: The kit of clause 3, wherein the blood collection devices each further comprise: a container engagement portion connected to the holder; and a collection container removably connectable to the container engagement portion, the container defining a collection cavity, wherein the actuation portion comprises at least two wings configured to create pressure for the patient's finger positioned within the finger receiving portion.

Clause 5: The kit of any of clauses 1-4, wherein at least 95% of the patient fingers for the population of patients are within one of the unique size ranges.

Clause 6: The kit of any of clauses 1-5, wherein the population of patients comprises a population of all adult patients living within a selected geographic region, and wherein the finger sizes for the population of patients substantially matches a normal distribution.

Clause 7: The kit of any of clauses 1-6, wherein a difference between a maximum value and a minimum value for each of the unique size ranges are equal to each other.

Clause 8: The kit of clause 7, wherein the unique ideal finger sizes for the plurality of blood collection devices are equally between the minimum value and the maximum value for each of the unique size ranges.

Clause 9: The kit of any of clauses 1-8, wherein the unique size ranges for the plurality of blood collection devices are selected such that an equal number of patient fingers of the patient population are within the unique size range for each of the blood collection devices of the plurality of blood collection devices.

Clause 10: The kit of any of clauses 1-9, wherein the unique ideal finger sizes for the plurality of blood collection devices are a minimum value of the unique size range for each of the plurality of blood collection devices.

Clause 11: The kit of any of clauses 1-9, wherein the unique ideal finger sizes for the plurality of blood collection devices is a maximum value of the unique size range for each of the plurality of blood collection devices.

Clause 12: The kit of any of clauses 1-9, wherein the unique ideal finger sizes for the plurality of blood collection devices is greater than a middle value of the unique size range for each of the plurality of blood collection devices.

Clause 13: The kit of any of clauses 1-9, wherein the unique ideal finger sizes for the plurality of blood collection devices are selected such that a ratio of loose/tight fit for finger sizes within the unique size ranges are the same for each of the plurality of blood collection devices.

Clause 14: The kit of any of clauses 1-13, wherein the kit comprises at least four blood collection devices of different sizes.

Clause 15: The kit of any of clauses 1-14, wherein the sizing tool comprises a sizing card for a size exclusion determination, the sizing card comprising a plurality of elliptical openings, wherein each of the plurality of elliptical openings is sized to correspond to a maximum value of the unique size range for one of the plurality of blood collection devices.

Clause 16: The kit of clause 15, wherein the plurality of elliptical openings each comprise a major diameter corresponding to a maximum finger width and a minor diameter corresponding to a maximum finger height for the unique size range for the one of the plurality of blood collection devices.

Clause 17: The kit of any of clauses 1-14, wherein the sizing tool comprises at least one measuring tool, such as a ruler or calipers, configured to directly measure at least one of a width, height, and/or length of the patient's finger.

Clause 18: The kit of any of clauses 1-14, wherein the sizing tool comprises a computer processor configured to: receive at least one image of the patient's finger; process the received at least one image to determine at least one of a finger width, height, and/or length of the patient's finger; and cause an output device to display an indication of which of the plurality of blood collection devices to use for the patient's finger determined based, at least in part, on the finger width, height, and/or length of the patient's finger determined by the processing of the at least one image and the unique size ranges for the plurality of blood collection devices.

Clause 19: A method of making a plurality of blood collection devices sized for use with at least a majority of patient finger sizes for a patient population, the method comprising: determining unique size ranges for each of the plurality of blood collection devices; determining a unique ideal size for each device of the plurality of blood collection devices, wherein the unique ideal size for each device is within the determined size range for the respective device of the plurality of blood collection devices; and making the plurality of blood collection devices with dimensions corresponding to the determined unique ideal size for each of the plurality of blood collection devices.

Clause 20: A computer-implemented method for determining a correct blood collection device size for a patient's finger, the method comprising: receiving, with at least one computer processor, at least one image of the patient's finger; processing, with the at least one computer processor, the received at least one image to determine at least one dimension of the patient's finger; and causing, with the at least one computer processor, an output device to provide a visual indication of which blood collection device of a plurality of blood collection devices having unique finger size ranges to use for the patient's finger determined based, at least in part, on the determined at least one dimension of the patient's finger and the unique size ranges for the plurality of blood collection devices.

### BRIEF DESCRIPTION OF THE DRAWINGS

FIG. 1A is a perspective view of a capillary blood collection device for obtaining a blood sample from a patient's finger and a collection container according to an aspect of the present disclosure.

FIG. 1B is a cross-sectional view of a capillary blood collection device and a lancet according to an aspect of the present disclosure.

FIG. 1C is a perspective view of a holder of a capillary blood collection device according to an aspect of the present disclosure.

FIG. 1D is a schematic drawing showing a top view of the holder of FIG. 1C connected to a patient's finger for performing a blood collection procedure.

FIG. 1E is another schematic drawing showing a front view of the holder of FIG. 1C connected to the patient's finger.

FIG. 2 is schematic drawing showing a set of blood collection devices of different sizes that can be used for capillary blood collection for a patient and sizing tools for identifying which blood collection device to use for the patient according to an aspect of the present disclosure.

FIG. 3 is an annotated graph showing exemplary device size ranges and ideal sizes for blood collection devices of a set of different sized devices according to an aspect of the present disclosure.

FIG. 4 is an annotated graph showing another example of device size ranges and ideal sizes for blood collection devices of a set of different sized devices according to an aspect of the present disclosure.

FIG. 5A is a flow chart showing steps of a computer implemented method for blood collection device sizing according to an aspect of the present disclosure.

FIG. 5B is a schematic drawing of a patient's finger that can be captured by a camera used for determining finger dimensions according to an aspect of the present disclosure.

FIG. 5C is an example of a screen that can be displayed on an output device showing a user what size blood collection device to use for a particular patient's finger according to an aspect of the present disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

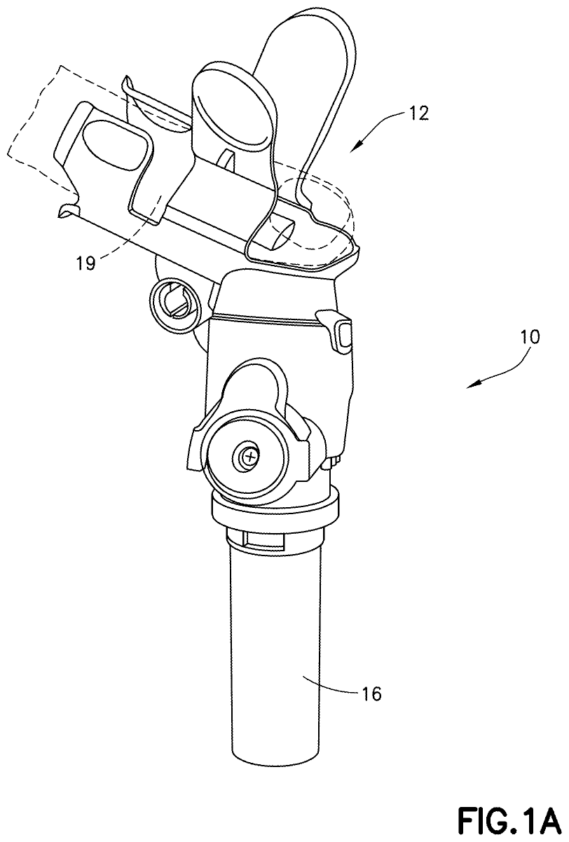

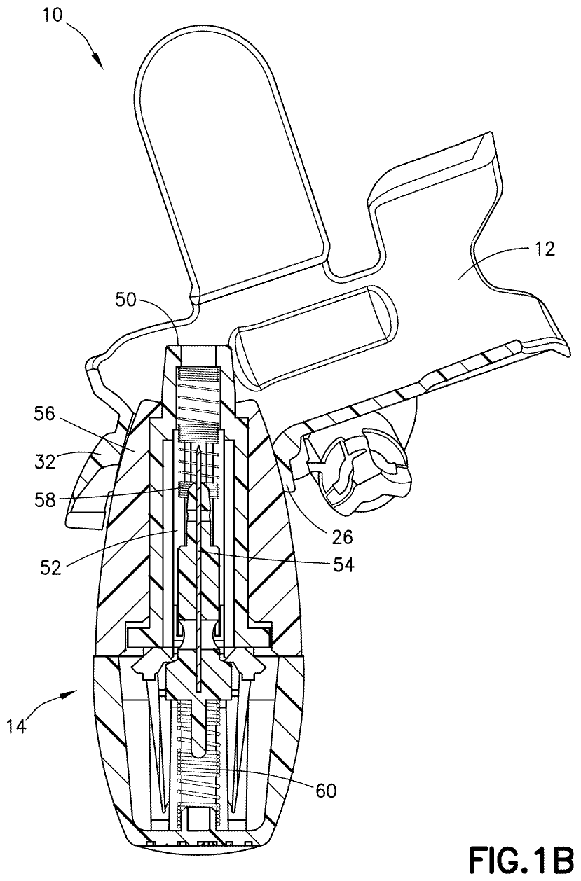

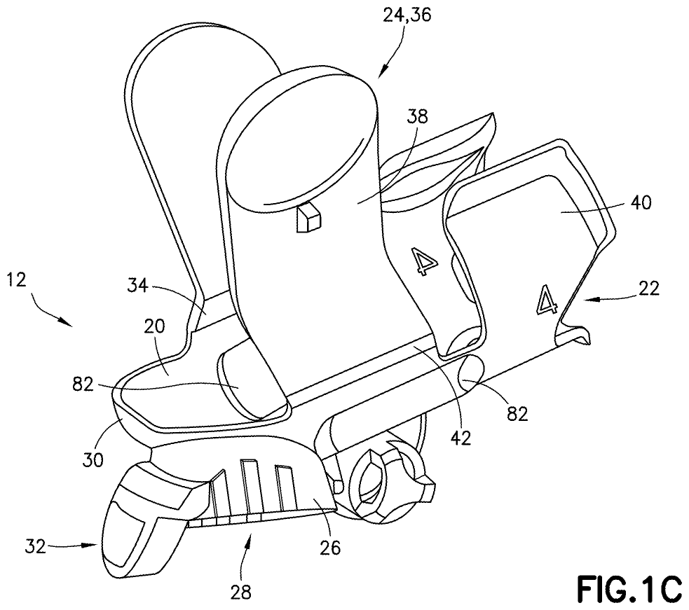

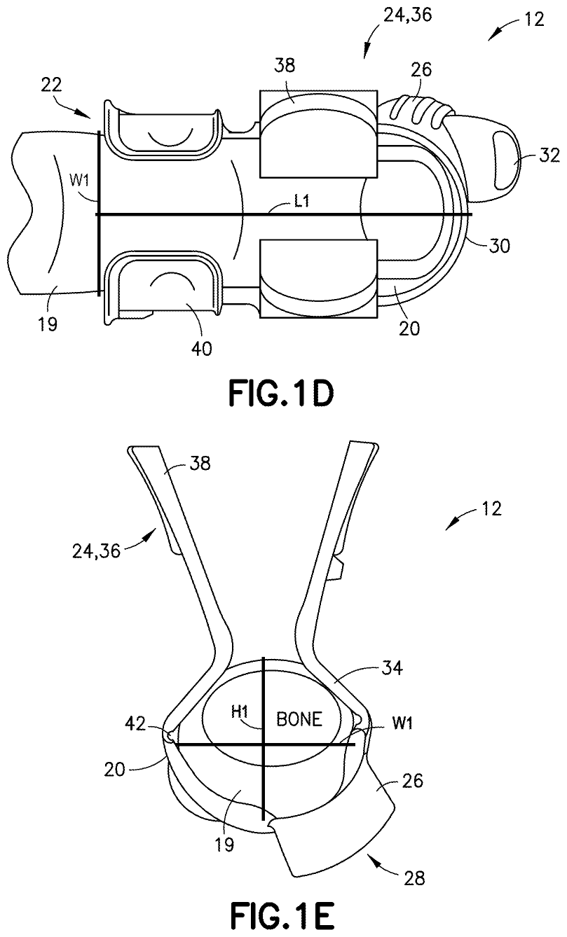

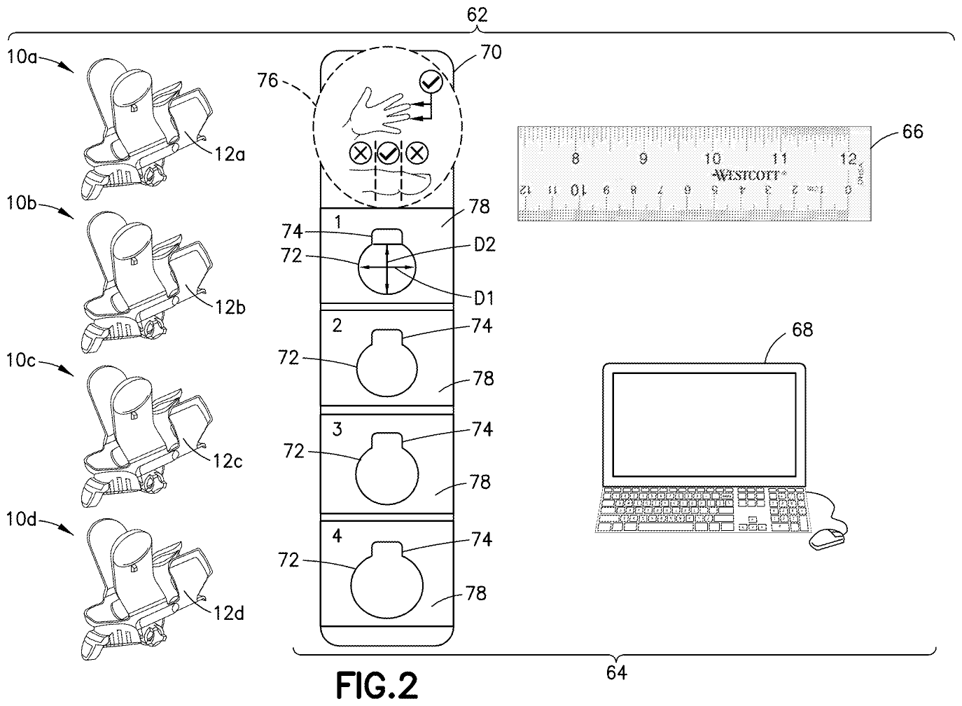

The present disclosure is directed to blood collection devices 10 (shown in FIGS. 1A-E) and/or sets or kits 62 (shown in FIG. 2) of blood collection devices 10 that are sized to correctly fit as many patients as possible. As will be appreciated by those skilled in the art, for many products that come in different sizes, such as clothing, there are typically no safety concerns with having multiple people try on the product for fit. Further, if there is a concern about proper sizing, a user or wearer can dispose of the product rather than continuing to use an incorrectly sized product. However, for medical devices, such as blood collection devices 10, there can be safety concerns with using a device 10 of an improper size for a patient's finger. Further, disposing of medical devices, such as a blood collection device 10, because the devices are not a suitable size for a particular patient is inefficient and increases costs of such devices and medical procedures using the devices 10. The devices, systems, and methods disclosed herein are intended to improve sizing for blood collection devices 10, as well as to help ensure that a correctly sized blood collection device 10 is being used for each patient.

According to one aspect, the present disclosure relates to methods for designing and/or manufacturing capillary blood collection devices 10 including finger cuffs or holders 12 that are sized to comfortably fit as many patients of a population (i.e., a normal distribution of adult finger and/or hand sizes) as possible in order to reduce occurrences of problems caused when capillary blood draw procedures are performed using incorrectly sized devices 10. For example, if the holder 12 or finger cuff is too loose, the holder 12 or cuff may move or fall off during a blood collection procedure. If the holder 12 or finger cuff is too tight, the device 10 may restrict blood flow or cause pain for the patient. As used herein, the “population” can refer to a defined subset of humans, such as finger sizes for all adults. As described herein, finger sizes for adults generally adhere to a normal distribution or bell-curve. The “population” can also refer to a subset of humans based on gender (i.e., all male adults), geographic location (i.e., all adults living in the United States), ethnicity (i.e., all Caucasian adults), or any other subset of a population, as may be selected by those skilled in the art.

According to another aspect, the present disclosure relates to the set or kit 62 of multiple blood collection devices 10 of different sizes (i.e., small, medium, large, and extra-large devices), wherein each of the devices 10 of the set or kit 62 is configured to be used for a unique size range of finger sizes (i.e., finger width, height, and/or length). As used herein, a “unique” size range for a device 10 of a set or kit 62 refers to a size range that is different from size ranges of other devices 10 of the set or kit 62. In some examples, the unique size ranges for the blood collection devices 10 of a set or kit 62 are entirely distinct, meaning that the unique size ranges for the devices 10 of the set or kit 62 do not overlap at all. In other examples, the unique size ranges for the devices 10 of the set or kit 62 may overlap by a small amount, such as by 5%, 10%, or 15%. Generally, the unique size ranges for the devices 10 of the set or kit 62 are continuous, meaning that there is no gap of finger sizes between, for example, a small sized device and a medium sized device. Further, desirably, the size ranges are selected so that one of the devices 10 of the set or kit 62 of devices 10 can be safely and comfortably used for, at least, a majority of possible finger sizes for the population (i.e., at least 50% of the population) or, preferably, for a substantial majority (i.e., at least 90%, 95%, 97.5%, or 99%) of the population.

According to another aspect, the present disclosure is also related to methods and measuring tools for determining correct sizing for a blood collection device 10, so that a user can determine which device 10 of the set or kit 62 of blood collection devices 10 to use for a particular patient's finger. The user can be, for example, a trained healthcare worker (i.e., a phlebotomist, nurse, or similarly trained medical professional), who is familiar with blood collection procedures. In other examples, the user can be a healthcare worker without blood collection experience, but who can perform minor procedures, such as a pharmacist or pharmacy technician. In other examples, the user can be the patient, who uses the blood collection device 10 for self-collection of a blood sample. The measuring tools can include, for example, manually operated measuring tools (i.e., rulers, calipers, and similar measuring devices), as well as automated methods based on processing of captured images by a computer, that determine exact dimensions for the patient's finger. The measuring tools can also include sizing cards, rings, loops, and similar implements that determine correct sizing based on size exclusion. As described in further detail herein, “size exclusion” can refer to a method in which correct sizing is determined by inserting the patient's finger through an opening corresponding to a minimum or maximum acceptable finger size for one of the devices 10 of the set or kit 62 of blood collection devices 10. If the patient's finger does not fit through the opening, then an opening corresponding to another of the devices 10 is tried. In this way, a user can determine what size device 10 to use for a particular patient's finger, without determining exact finger dimensions of the patient's finger.

### Blood Collection Device

Examples of a blood collection devices 10 having dimensions that can be optimized to correctly fit patients within defined size ranges are shown in FIGS. 1A-1E. The blood collection device 10 can be, for example, a self-contained and fully integrated finger-based capillary blood collection device with the ability to lance, collect, and stabilize a high volume capillary blood sample, e.g., up to or above 500 microliters. The blood collection device 10 can also be a device formed from separable components (i.e., a finger cuff or holder 12, lance, and sample container) that can be connected and/or used together to obtain a blood sample. Some additional exemplary capillary blood collection devices and assemblies that can be modified for use with the systems and methods of the present disclosure are described in United States Patent Appl. Pub. No. 2019/0216380, entitled “Device for Obtaining a Blood Sample”; United States Patent Appl. Pub. No. 2019/0223772, entitled “Device for the Attached Flow of Blood”; and PCT Publication No. WO 2020/167746, entitled “Capillary collector with rotatable connection,” each of which is incorporated herein by reference in its entirety.

With reference to FIGS. 1A and 1B, the example blood collection device 10 includes the integrated holder 12 (also referred to herein as the finger cuff), a lancet housing or lancet 14 (shown in FIG. 1B) for puncturing a finger 19 (shown in FIGS. 1D and 1E) of the patient, and a collection container 16 (shown in FIG. 1A). In other examples, the blood collection device 10 can be provided as a semi-integrated device 10 including, for example, an integrated lancet housing 14 and collection container 16 that can be connected with a separate holder 12. In other examples, a semi-integrated device may have an in-line flow and an integrated lancet housing and collection container, which can be connected with a separate holder.

The holder 12 is configured to receive a sample source, e.g., the finger 19 of a patient, for supplying a biological sample, such as a blood sample. As shown in FIGS. 1C-1E, the holder 12 generally includes a finger receiving portion 20 having a first opening 22, an actuation portion 24, a port 26 having a second opening 28, and a finger end guard 30. The first opening 22 can have a width W1 (shown in FIGS. 1D and 1E), which corresponds to a maximum finger width for a finger 19 that can be used with the device 10. The first opening 22 can also have a height H1 (shown in FIG. 1E), which corresponds to a maximum finger height for a finger 19 that can be used with the device 10. The finger receiving portion 20 can also have a length L1 (shown in FIG. 1D), which corresponds to a maximum finger length (i.e., finger length from the fingertip to the second knuckle) of a finger 19 that can be used with the device 10. If a patient's finger 19 is greater than the width W1, height H1, and/or length L1, then a different size of blood collection device 10 and/or holder 12 should be used for the patient.

The finger end guard 30 is configured to provide a stop portion for properly aligning and securing the finger 19 within the holder 12. The finger end guard 30 further assists in ensuring the patient's finger 19 is placed at a proper position within the finger receiving portion 20 so that applied pressure to the patient's finger 19 will result in adequate blood flow. The finger end guard 30 can have a curved fingertip rest that ensures the patient's finger 19 stops at an end of the finger receiving portion 20, while permitting the patient's finger nail to clear the end of the finger receiving portion 20. The finger receiving portion 20 permits use of the holder 12 with artificial and natural fingernail styles present in the patient population.

The first opening 22, having the width W1 and the height H1, is configured for receiving the sample source, e.g., the finger 19. The sample source may also include other parts of the body capable of fitting within the first opening 22, such as toes or other extremities. The port 26 is in communication with the finger receiving portion 20. For example, with a finger 19 received within the holder 12, the port 26 is in communication with a portion of the finger 19. As described in further detail herein, the holder 12 can be sized for use with a particular subset of patients. For example, a small holder 12 may be an appropriate size of a bottom quartile of patients. Medium and large-sized holders 12 can be sized for use with patients in the lower middle quartile of patients (25% to 50%) and the upper middle quartile of patients (50% to 75%), respectively.

The second opening 28 of the port 26 is configured for receiving the lancet housing or lancet 14 (shown in FIG. 1B) and a collection container 16 (shown in FIG. 1A). In some examples, the port 26 further includes a locking portion 32 for securely receiving the lancet housing or lancet 14 and the collection container 16 within the port 26.

The actuation portion 24 of the device 10 is transitionable between a first position, in which the holder 12 defines a first diameter, and a second position, in which the holder 12 defines a second diameter, with the second diameter being less than the first diameter. Further, in the first position, the holder 12 defines a first elliptical shape. In the second position, the holder 12 defines a second elliptical shape, with the first elliptical shape being different than the second elliptical shape. In this manner, with the holder 12 in the second position with a reduced diameter, a portion of the holder 12 contacts the sample source (i.e., the finger 19) and the actuation portion 24 of the holder 12 is able to pump and/or extract blood as described in more detail below.

In some examples, the actuation portion 24 includes a contact member 34. With the actuation portion 24 in the first position, the contact member 34 is in a disengaged position, i.e., the contact member 34 is provided in a first position with respect to the sample source, such that the contact member 34 may be in slight contact therewith. With the actuation portion 24 in the second position, the contact member 34 is in an engaged position, i.e., the contact member 34 is provided in a second position with respect to the finger 19, such that the contact member 34 is in an applied pressure contact with the finger 19, and the actuation portion 24 of the holder 12 is able to pump and/or extract blood. For example, with the contact member 34 in the engaged position, the contact member 34 exerts a pressure on the sample source.

In some examples, the actuation portion 24 includes a pumping member 36 for applying pressure to the finger 19, such as a pair of opposed tabs or wings 38. Each wing 38 can include a contact member 34. The holder 12 can also include a living hinge portion 42. The living hinge portion 42 allows the user to squeeze the wings 38 between a first position (passive state) and a second position (active state). It is believed that use of the tabs or wings 38 to draw blood out of a patient's finger 19 minimizes hemolysis while maintaining an adequate flow of blood from the patient's finger 19. A resting position and hinge of the wings 38 are designed to maintain contact and retention with the smallest patient finger that can fit into a holder 12, while flexing to accommodate the largest patient finger within a holder 12 without blood occlusion. In some examples, the wings 38 may be positioned on the finger receiving portion 20 at a position located proximal of a patient's fingernail and distal of a patient's first knuckle to avoid hard tissues on the patient's finger 19.

The holder 12 can be configured to allow a user to repeatedly squeeze and release the wings 38 to pump and/or extract blood from a finger 19 until a desired amount of blood is filled in a collection container 16. The wings 38 are configured to flex to maintain gentle contact with a range of patient finger sizes that may be used with the holder 12 and to retain the holder 12 on the patient's finger 19. The wings 38 may also provide active pressure features for the holder 12.

In some examples, the holder 12 can include a stability extension portion 40. This provides additional support for the holder 12 to be securely placed onto the finger 19. In one example, the finger receiving portion 20 forms a generally C-shaped member and includes a plurality of inner gripping members for providing additional grip and support for the holder 12 to be securely placed onto a finger 19. The stability extension portion 40 assists in maintaining contact with the patient's finger 19 during use of the holder 12 while avoiding the blood supply and knuckles of the patient's finger 19.

The blood collection device 10 for obtaining the blood sample also includes the lancet housing or lancet 14 (shown in FIG. 1B) that is removably connectable to the port 26 of the holder 12. Referring to FIG. 1B, the lancet housing or lancet 14 can include an inlet or opening 50, an interior 52, a puncturing element 54, an engagement portion 56, a retractable mechanism 58, and a drive spring 60. The puncturing element 54 can be moveable between a pre-actuated position, wherein the puncturing element 54 is retained within the interior 52 of the lancet housing 14, and a puncturing position, wherein at least a portion of the puncturing element 54 extends through the inlet 50 of the lancet housing or lancet 14 to lance a portion of a finger 19. In one example, the lancet 14 of the present disclosure is a contact activated lancet and may be constructed in accordance with the features disclosed in U.S. Pat. No. 9,380,975, entitled “Contact Activated Lancet Device”, which is incorporated herein by reference in its entirety.

In some examples, the holder 12 and the lancet housing or lancet 14 are separate components that can be removably connectable to the port 26 of the holder 12. In such examples, the lancet housing or lancet 14 includes the engagement portion 56. The lancet housing or lancet 14 can be pushed into the port 26 of the holder 12, such that the engagement portion 56 of the lancet housing or lancet 14 is locked within the locking portion 32 of the holder 12. In this manner, the lancet housing 14 is securely connected and locked to the holder 12, such that the puncturing element 54 of the lancet housing 14 can be activated to lance or puncture a sample source, e.g., the finger 19. In some examples, the port 26 of the holder 12 includes a plurality of ribs for securing and locking the lancet 14 or the collection container 16 in the port 26.

To activate the lancet 14, the lancet 14 is pushed against the finger 19 to activate the retractable mechanism 58 and drive spring 60 of the lancet 14 to lance the finger 19. After puncturing, the puncturing element 54 is immediately retracted and safely secured within the interior 52 of the lancet housing 14. Once the finger 19 is punctured, the blood sample is squeezed from the finger 19 into a collection container 16. The collection container 16 may also contain a sample stabilizer, e.g., an anticoagulant, to stabilize a blood sample and/or a component of a blood sample disposed therein. The collection container 16 may also include at least one fill line(s) corresponding to a predetermined volume of sample. The collection container 16 may also indicate/meter a collected volume of blood.

In order to use the capillary blood collection device 10 shown in FIGS. 1A-1E, a desired finger 19 is first cleaned and a holder 12 having an appropriate size for the desired finger 19 is selected and placed onto the finger 19 securely. Next, the lancet housing or lancet 14 is connected to the port 26 of the holder 12. As discussed previously, the lancet housing or lancet 14 is pushed into the port 26 of the holder 12, such that the engagement portion 56 of the lancet housing or lancet 14 is locked within the locking portion 32 of the holder 12. In this manner, the lancet housing or lancet 14 is securely connected and locked to the holder 12, such that the puncturing element 54 of the lancet housing 14 can be activated to lance or puncture the finger 19. With the lancet 14 connected to the port 26 of the holder 12, the lancet 14 is in communication with the finger 19.

When it is desired to activate the lancet 14 to lance the skin of the finger 19, the lancet 14 is pushed against a finger 19 to activate a retractable mechanism 58 of the lancet 14 to lance the finger 19. After the finger 19 is lanced to create blood flow from the finger 19, the lancet 14 is removed from the holder 12 and the collection container 16 is pushed into the port 26 of the holder 12. With the container 16 properly secured to the holder 12 for collection of a blood sample, the user repeatedly squeezes and releases the wings 38 of the holder 12 to pump and/or extract blood from the finger 19 until a desired amount of blood is collected in the collection container 16. Advantageously, with the holder 12 placed onto a finger 19, the holder 12 does not constrict the blood flow and defines lancing and finger squeezing locations. The squeezing tabs or wings 38 provide a pre-defined range of squeezing pressure that is consistently applied throughout a finger 19. By doing so, the holder 12 provides a gentle controlled finger massage that stimulates blood extraction and minimizes any potential hemolysis.

Once a desired amount of blood is collected within the container 16, a blood collector portion including the collection container 16 can be detached from the collection device 10 in order to send a collected sample to a diagnostic instrument and/or testing device. The blood collector portion can be sealed via the cap or septum once removed from the collection device 10 to protectively seal the blood sample within the collection container 16.

### Multi-Size Capillary Blood Collection Set or Kit

The previously described holder 12 and blood collection device 10 provide advantages over conventional capillary blood collection devices. In particular, the holder 12 is configured to align with a patient's finger features, ensuring that the holder 12 consistently and securely remains in place and applies pressure in the correct location. This feature was accomplished by analyzing several sources of anatomical information (finger width and length, knuckle and artery locations) to limit squeezing to soft tissues near the collection site while avoiding pressure on hard tissues or blood vessels. Further, the wings 38 are configured to apply pressure in two stages. The first stage has pressure on the finger increased proportionally to the applied pressure. However, as intensity increases, the wings 38 begin to flex and bend until they touch and cannot displace any further. This two-stage application of pressure allows enough pressure to have adequate blood flow but limits maximum pressure to avoid hemolysis.

In order to achieve such benefits in how pressure is applied to the finger 19 during the blood collection procedure, it is important that the holder 12 is sized correctly for the patient's finger, meaning that the holder 12 is neither too loose nor too tight to function properly. For example, if the holder 12 is too loose, the holder 12 may move relative to or fall off from the finger 19 during a blood draw procedure. If the holder 12 is too tight, the holder 12 may restrict blood flow or cause pain for the patient.

Since it is impractical to produce unlimited sizes of holders 12, the set or kit 62 includes a discrete number of holders 12 of different sizes. For examples, as shown in FIG. 2, the set or kit 62 of blood collection devices 10a, 10b, 10c, 10d, can include a device 10a with a small-sized holder 12a, a device 10b with a medium-sized holder 12b, a device 10c with a large-sized holder 12c, and a device 10d with an extra-large sized holder 12d. The different sized devices 10a, 10b, 10c, 10d are designed to fit different unique size ranges of finger sizes. For example, the different sizes of devices 10a, 10b, 10c, 10d can be made to accommodate individuals with different finger lengths (i.e., a distance between the fingertip and the second knuckle of the finger being used for the puncture procedure) and/or different finger widths and/or heights. In particular, sizes of different components of the devices 10a, 10b, 10c, 10d, such as a width W1 and/or height H1 of the first opening 22 and/or length L1 of the finger receiving portion 20, can be different for each size of device 10a, 10b, 10c, 10d to accommodate a specific size range.

As previously discussed, the unique size ranges of the devices 10a, 10b, 10c, 10d are selected such that a majority or, preferably, a substantial majority of fingers for a population of patients are within one of the unique size ranges for the blood collection devices 10a, 10b, 10c, 10d. Further, as described in greater detail hereinafter, the unique size ranges for the different sized devices 10a, 10b, 10c, 10d can be optimized based, for example, on a normal population distribution of finger sizes, to ensure that one of the devices 10a, 10b, 10c, 10d comfortably fits as many finger sizes within the population as possible.

With continued reference to FIG. 2, the set or kit 62 can also include a sizing tool 64 for identifying which of the plurality of blood collection devices 10a, 10b, 10c, 10d to use for a particular patient's finger 19. In some examples, the sizing tool 64 is a measuring tool 66, such as a ruler or calipers, for directly measuring dimensions of the patient's finger 19. In particular, measuring tools 66 are configured to directly contact the patient's finger 19 to obtain measurements (i.e., width, height, and/or length) for the patient's finger 19. In other examples, the sizing tool 64 can be a computer device 68 that includes a computer processor which, as described in further detail hereinafter, processes images of the patient's finger 19 to determine dimensions of the patient's finger 19. In other examples, as described in further detail hereinafter, the sizing tool 64 is a sizing card 70 configured to determine proper sizing information for the patient's finger 19 by size exclusion.

### Methods for Optimizing Size Ranges

Dimensions for the different sizes of the devices 10a, 10b, 10c, 10d can be determined, for example, from size charts and other anatomical data for average sized adult and/or pediatric patients. For example, the smallest device 10a can be sized to accommodate a finger width, height, and/or length corresponding to 25^{th} percentile for an average adult person. Similarly, the medium device 10b can be sized for persons falling between the 25^{th} and 50^{th} percentiles for hand and/or finger sizes; the large device 10c can be sized for persons with a hand and/or finger size falling between the 50^{th} percentile and the 75^{th} percentile; and the extra-large device 10d can be sized for persons with a finger size falling between the 75^{th} percentile and the 100^{th} percentile. In some examples, size ranges for the devices 10a, 10b, 10c, 10d can be further optimized, for example, to minimize a mismatch between the holder size and a person's finger size for the largest possible number of individuals of the population. In other examples, size ranges may be optimized so that a substantially equal number of persons within a population use each size of the device 10a, 10b, 10c, 10d.

The present inventors have determined numerous methods for determining the unique size ranges for each of the blood collection devices 10a, 10b, 10c, 10d of the set 62, as well as for determining dimensions (i.e., width W1, height H1, and/or length L1) of the holders 12a, 12b, 12c, 12d for each blood collection device 10a, 10b, 10c, 10d of the set 62.

One simple solution to determine sizing is to determine the range in human finger dimensions from smallest to largest for the entire population of patients and divide the range equally based on the total number of devices 10a, 10b, 10c, 10d in the set or kit 62. For example, if the range of expected finger widths for the population is 10.0 mm to 20.0 mm, and if the set 62 includes four different sized capillary blood collection devices 10a, 10b, 10c, 10d, then the size range (i.e., the difference between the maximum value and the minimum value for each unique size range) can be 2.5 mm for each blood collection device 10a, 10b, 10c, 10d. For example, the size ranges for finger widths can be: small (10 mm to 12.5 mm); medium (12.6 mm to 15.0 mm); large (15.1 mm to 17.5 mm); and extra-large (17.6 mm to 20.0 mm). Further, each device 10a, 10b, 10c, 10d can be designed to exact dimensions, referred to herein as the “unique ideal finger size” dimensions, for a finger having a width that is in the middle of each unique size range for each device 10a, 10b, 10c, 10d. Specifically, the small device 10a can be designed for a unique ideal finger width of 11.25 mm. The medium device 10b can be designed for a unique ideal finger width of 13.75 mm. The large device 10c can be designed for a unique ideal finger width of 16.25 mm. The extra-large device 10d can be designed for a unique ideal finger width of 18.75 mm. This sizing distribution ensures that for any finger width in the entire population (10.0 mm to 20.0 mm) the patient's actual finger width is within ±1.25 mm of the unique ideal finger width for one of the devices 10a, 10b, 10c, 10d. Accordingly, it is more likely that each patient will find a suitable device than if a single universal device was used for all patients and finger widths. Further, as will be appreciated by those skilled in the art, the same process could be performed to design devices 10a, 10b, 10c, 10d for size ranges of finger height and/or finger length, within the scope of the present disclosure.

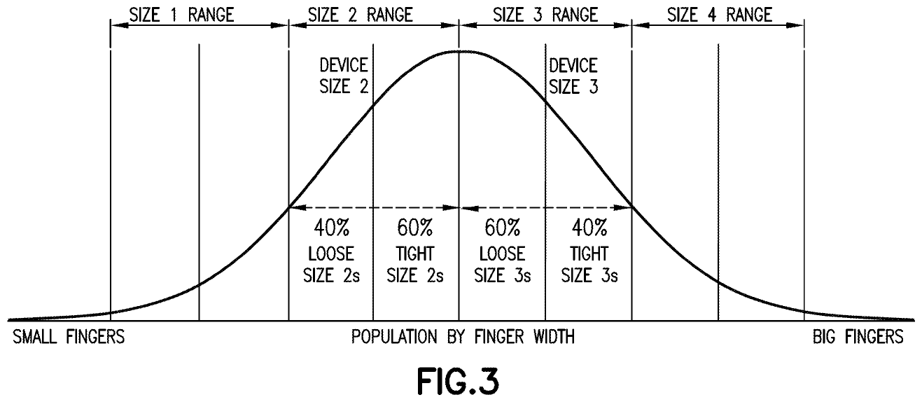

With reference to FIG. 3, a drawback to this simple approach of determining the size range and ideal finger size for each device 10a, 10b, 10c, 10d is that finger sizes for a population generally follow a normal distribution or bell-curve, meaning that a majority of patient finger sizes (width, height, and/or length) are close to the mean average (50^{th} percentile) finger size. Therefore, when a total finger size range for a population is divided into three or four unique size ranges for different devices 10a, 10b, 10c, 10d, the majority of patients use the middle size(s) of devices and fewer patients use the outside (i.e., the smallest or largest) sizes of devices.

Further, when this simple approach to determining the unique size ranges and the unique ideal size for each device 10a, 10b, 10c, 10d is applied, the percentage of patients with loose or tight fit will vary for different devices 10a, 10b, 10c, 10d of the set or kit 62. For example, as shown in FIG. 3, for the medium device 10b, the device 10b is tight for 60% of users and is loose for 40% of users. In contrast, for the large device 10c, the device 10c is loose for 60% of users and tight for 40% of users. This inconsistent sizing experience may confuse users, such as healthcare workers, creating a perception that certain sizes are tight or loose and need to be adjusted outside of size range recommendations. For example, users may come to assume that the medium device 10b is often too tight for patients, leading the users to use the large device 10c for some patients, when such sizing is not actually appropriate.

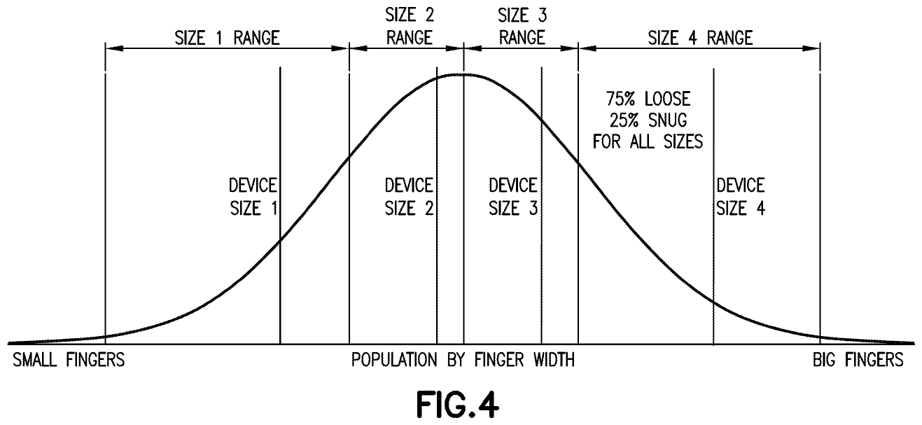

With reference to FIG. 4, another method for determining the unique size ranges and unique ideal finger sizes (width, height, and/or length) for each device 10a, 10b, 10c, 10d involves an optimization algorithm based on a distribution of sizes weighted by population to achieve a better fit for the majority of patients. As shown in FIG. 4, the optimization method produces size ranges for the middle devices (i.e., the medium device 10b and the large device 10c) that are smaller (i.e., a difference between a maximum value and a minimum value for the size range is smaller) than for the outside devices (i.e., the small device 10a and the extra-large device 10d). Specifically, the unique size ranges for each device 10a, 10b, 10c, 10d can be optimized so that a same number of persons (i.e., 25% of the population) or substantially the same number of persons of the population (i.e., 20% to 30% of the population) will use each device 10a, 10b, 10c, 10d of the set 62. Beneficially, modifying the size ranges in this manner ensures that patients are equally or substantially equally distributed across the device sizes, allowing more balanced production and use of the device sizes.

Also, as shown in FIG. 4, the ideal finger size for each device 10a, 10b, 10c, 10d is not in the middle of the size range, as was the case in FIG. 3. Instead, the unique ideal finger size for each device 10a, 10b, 10c, 10d is weighted based on population distribution and so that a ratio of loose/tight fit is the same for all devices 10a, 10b, 10c, 10d. For example, as shown in FIG. 4, the unique ideal finger size for each device 10a, 10b, 10c, 10d can be selected so that each device 10a, 10b, 10c, 10d is loose for 75% of the size range and tight for 25% of the finger sizes within each size range. In contrast, in the example of FIG. 3, the ratio of loose/tight fit was different for each device 10a, 10b, 10c, 10d, which may cause confusion for users, such as healthcare workers, and/or may cause users not to follow sizing recommendations provided by the sizing tools and methods of the present disclosure.

Optimization of the size ranges and ideal finger sizes for the devices 10a, 10b, 10c. 10d can be accomplished by an iterative method and penalty function based on mis-fit between any randomly selected finger size and the ideal finger size. In some examples, the calculated mis-fit can be weighted based on population distribution. Further, the optimization can occur with additional solution constraints, such as preventing a mis-fit greater than a certain limit for each size range and/or enforcing a loose/tight ratio, as previously described. Beneficially, the optimization method balances size ranges by population and fit, giving good fit to the maximum number of possible patients. Further, the optimization method can balance the unique ideal finger size for each device to ensure each size has a similar population distribution of loose and tight fits.

More specifically, the optimization method involves calculating how much the random finger differs in size from the ideal finger size for a particular blood collection device. Using this approach, a mis-fit penalty is calculated for every possible finger size within each size range. As used herein, the “mis-fit” refers to the difference between a dimension (finger width, height, and/or length) of the randomly selected finger size and the holder 12a, 12b, 12c, 12d. The mis-fit penalty can be calculated in different ways. For example, the mis-fit penalty can be based on absolute mis-fit for each random finger size within the range. The mis-fit penalty can also be based on a population-weighted mis-fit that takes into account both the absolute mis-fit for each random finger size and the frequency of the random finger size within the population. Thus, the population weighted mis-fit penalty may be large for small differences between a random finger size and the ideal finger size, when the random finger size is very common within a population. The population weighted mis-fit penalty may be relatively small when there is a large difference between a random finger size and the ideal finger size, provided that the random finger size occurs infrequently in the population.

In order to optimize the unique size range and unique ideal finger size for each device 10a, 10b, 10c, 10d of the set or kit 62, the mathematical algorithm or approach seeks to minimize the mis-fit penalty for all possible finger sizes within each size range. The end result for such optimization is desirably to provide a set or kit 62 of blood collection devices 10a, 10b, 10c, 10d of different sizes to ensure that: (i) every finger does not exceed a certain maximum level of mis-fit for at least one of the available device sizes; (ii) a majority of patient finger sizes within each size range are as close to the ideal finger size for the size range as possible; (iii) the percentage of patients with loose or tight fit is the same across all device sizes for a consistent collection experience; and/or (iv) each size of device achieves a blend of a maximum and/or average mis-fit.

In some examples, optimization of size ranges can be calculated according to the following steps of an optimization loop: (i) set and/or adjust the unique size ranges for each device 10a, 10b, 10c, 10d of the set 62; (ii) set and/or adjust the unique ideal finger size for each device 10a, 10b, 10c, 10d of the set 62; (iii) calculate mis-fit for all possible finger sizes within each size range; (iv) multiply each calculated mis-fit by finger size likelihood (based on population distribution); (v) sum across all possible finger sizes within the size range to calculate mis-fit penalty for the size range; (vi) sum mis-fit penalties to determine total mis-fit penalty for all devices 10a, 10b, 10c, 10d of the set or kit 62; (vii) compare the total mis-fit penalty for all devices 10a, 10b, 10c, 10d of the set or kit 62 to a pervious iteration of the optimization loop; and (viii) if the difference between the current iteration and the previous iteration is small (i.e., less than a predetermined value), the optimization loop is completed, or, if the difference between the current iteration and the previous iteration is large (i.e., greater than the predetermined value), adjust the loop inputs (size range and/or ideal finger size for each range) and rerun the optimization loop.

The optimization loop or model can be represented mathematically by a summation equation including the following inputs: a distribution of the finger measurement of interest (e.g. finger width, height, and/or length) for a population; percentage of the distribution the model will cover (e.g. exclude smallest and largest 2% of fingers for the population); a number of device sizes/ranges that will be included in the set or kit 62; and, optionally, any constraints on the ideal finger size for each device.

In some examples, the mathematical optimization model can be driven by the following basis equation:

$${\sum }_{n = S i z e 1}^{Max S i z e} [ {\sum }_{x = D e v i c e N R a n geM i n}^{D e v i c e N R a n geMa x} ( D e v i c e S i z {e}_{n} - x ) * P D F ( x ) ]$$

The variables in the above equation refer to the following inputs: ideal finger size for each device (DeviceSize); random finger size (x) within the size range for each device; and the probability for a particular finger size based on a finger size distribution (PDF(x)) for a population. As this function shows, the calculated mis-fit summation is weighted based on population distribution to optimize fit for the greatest number of patients. Alternatively, the distribution can be evenly weighted to instead penalize absolute differences between the device and fingers, or according to any other criteria, by adjusting the finger distribution.

The mis-fit between a device size and patient fingers is summed across the sizing range for that device (DeviceNRangeMin to DeviceNRangeMax). The same calculation occurs for the desired number of sizes and is summed across sizes (Size1 to MaxSize). This grand summation is the initial starting value for mis-fit. The model then iteratively updates the device size and device size range to minimize the grand total, typically using a Newton-Raphson, Levenberg-Marquadt, or other optimization technique for continuous functions. When the model converges on the lowest total mis-fit, the sizes have been optimized for best fit given the input criteria.

As previously described, this model can also be further constrained by other controlling functions. In some examples, the unique ideal finger size for each device 10a, 10b, 10c. 10d is defined at a particular position within the size range for the device. For example, the ideal finger size can be placed at the maximum of the range (all fingers will be loose fit), at the minimum of the range (all fingers will be tight fit), at a defined ratio of loose/tight fit based on population distribution, or any other criteria. As previously described, the model could be optimized so that the device 10a. 10b, 10c, 10d is tight for 25% of the population and loose for 75% of the population for each size range and device 10a, 10b, 10c, 10d. The model will then only be able to adjust the size ranges during optimization and ideal finger size will be automatically placed based on the criteria. The following equation can be used for calculating loose fit percentage for each possible finger size within a size range.

$$\frac{\sum [ PDF ( D e v i c e S i z {e}_{n} ) - P D F ( D e v i c e N R a n geM i n ) ]}{\sum [ PDF ( D e v i c e N R a n geMa x ) - P D F ( D e v i c e N R a n geM i n ) ]} = \% Loose Fits$$

The model can also be constrained to limit an absolute device mis-fit for each possible finger size within a range. For example, for a finger width size range of from 12.5 mm to 15.0 mm, the model may be constrained so that the maximum possible mis-fit is 2.0 mm, regardless of what the population weighted model for the ideal finger size determines. Constraining the absolute mis-fit means that for all patients, at least a minimum level of comfort will be achieved.

### Sizing Tools, Techniques, and Systems

It is expected that a healthcare worker or another user will be responsible for correctly sizing a patient's finger to determine which blood collection device 10a, 10b, 10c, 10d of the set or kit 62 to use for a particular blood draw procedure. The present inventors have identified a number of different devices, methods, and systems for assisting users to correctly size the patient's finger 19 to determine which device 10a, 10b, 10c, 10d of the set or kit 62 to use for a particular patient.

A simple method for device sizing is a contact measurement method in which the user directly determines finger size parameters to determine which blood collection device 10a, 10b, 10c, 10d to use for a particular patient. For example, the user may use the measuring tool 66 (i.e., a ruler, calipers, or any other measuring tool) to directly measure specific locations of the patient's finger 19. Once the particular dimensions of the patient's finger 19 are known, the user can determine which device 10a, 10b, 10c, 10d of the set 62 to use for the patient based on the determined (i.e., calculated and/or optimized) size range for each device 10a, 10b, 10c. 10d.

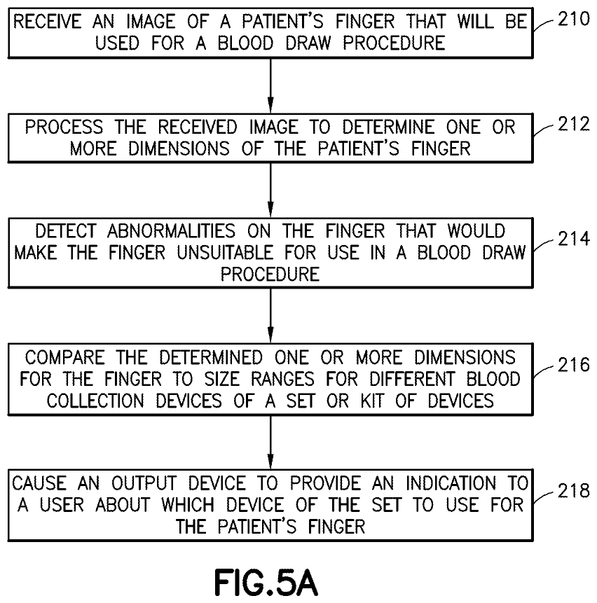

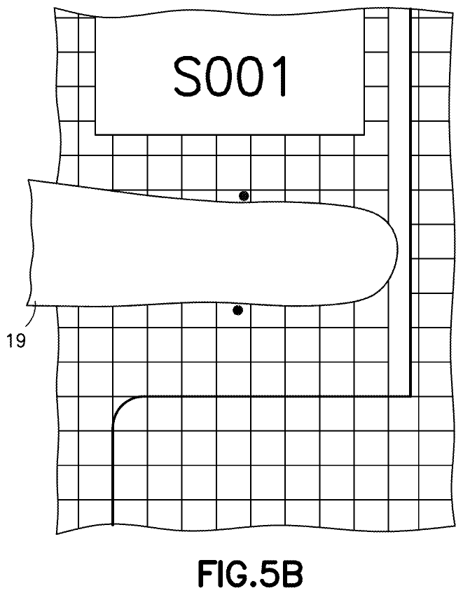

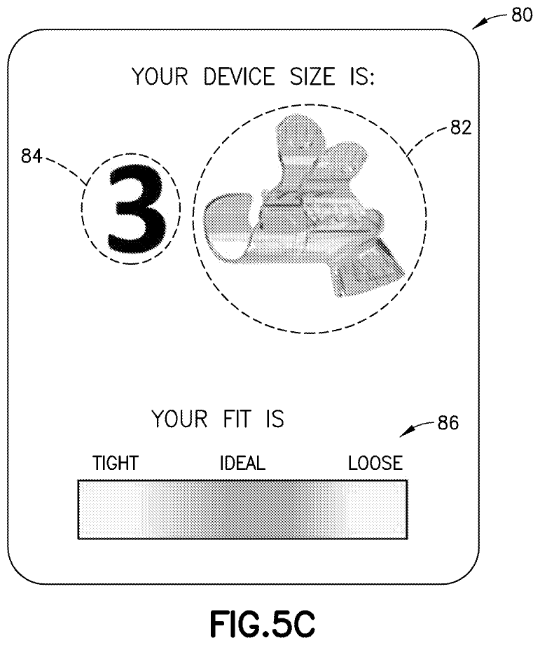

In other examples, a sizing method or algorithm could be performed by the computer device 68 to determine correct sizing. A flow chart showing a computer process for determining sizing is shown in FIG. 5A. As shown in FIG. 5A, at step 210, a computer processor receives an image of a patient's finger that will be punctured to perform a blood draw procedure. The image can be a digital image captured by a digital camera, such as a camera associate with a smart phone or tablet computer, as are known in the art. An exemplary image that may be provided to the computer processor is shown in FIG. 5B. As shown in FIG. 5B, the finger 19 is positioned on graph paper to assist in determining dimensions of the finger.

At step 212, the computer processor processes the image to determine one or more dimensions of the finger, such as the width, height, and/or length of the finger. Various image processing techniques will be known to those skilled in the art for determining dimensions of the finger captured in the digital image. For example, image processing can include processing the image to identify pixels in the image showing the finger and determining a number of pixels for width and/or length of the finger. Image processing can also include identifying other objects in the image, such as boxes of the graph paper, having a known size and determining a number of pixels corresponding to a height or width of the box. Based on this pixel information, the computer processor can determine the finger dimensions.

At step 214, processing the image can further include detecting abnormalities on the finger that would make the finger unsuitable for use in a blood draw procedure. For example, the computer processor may process the image to identify cuts, bruises, or scrapes on the finger. If the identified cuts, bruises, or scrapes are determined to be significant, the computer processor may output a warning and/or instruction asking the user to select another finger to use for the blood collection procedure.

At step 216, the one or more dimensions for the finger are compared to size ranges for the different devices 10a, 10b, 10c, 10d of the set 62 to determine which device 10a, 10b, 10c, 10d is a correct fit for the patient's finger. The finger dimensions can also be compared to the ideal finger size for the selected device. Based on this comparison, it can be determined whether the selected device 10a, 10b, 10c, 10d is tight or loose compared to the unique ideal finger size for each device.

At step 218, once the correct device is determined, the computer processor can cause an output device to provide an indication to a user (e.g., the healthcare worker or another user who will be performing the blood draw procedure) about which device of the set 62 to use. For example, the output device can be a visual display of a computer or a portable electronic device, such as a smartphone or computer tablet. An exemplary screen 80 that can be shown to the user is provided in FIG. 5C. As shown in FIG. 5C, the screen 80 includes an icon or image 82 of the selected device. The screen 80 can also include a numerical representation 84 identifying the selected device. In some examples, the screen 80 also includes a visual indication 86 showing correspondence between the determined dimensions of the patient's finger and the unique ideal finger size of the selected device. This visual indication 86 can be used to let the patient know whether the selected device may feel tight or loose, which can help to manage patient expectations about how the selected device will feel when placed on the patient's finger. The screen 80 can also include warnings about any cuts or scrapes on the patient's finger detected by the image processing and/or recommendations that another finger should be used for the blood draw procedure.

With reference again to FIG. 2, another option for determining device sizing is by a size exclusion method, which compares a size of a finger to be punctured to a minimum or maximum acceptable size (i.e., maximum finger width, height, and/or length) for each of the sizes of blood collection devices 10a, 10b, 10c, 10d. As previously described, size exclusion refers to tests for comparing the patient's finger to the minimum or maximum acceptable size for each range to determine proper fit without determining actual dimensions (e.g., finger width, height, and/or length) for the patient's finger. For example, the patient's finger can be inserted through an opening or another feature corresponding to a maximum size for the smallest device 10a. If the patient's finger cannot fit through the opening or feature, the patient's finger is too big for the small blood collection device 10a. In that case, the patient should attempt to pass his or her finger through openings or features of other devices 10b, 10c, 10d until finding the appropriate device size.

In some examples, a size exclusion test can be performed using a sizing card 70. The sizing card 70 can be, for example, a flat card including elliptical openings 72 sized to correspond to the different sizes of blood collection devices 10a, 10b, 10c, 10d of the set 62. The card 70 can be formed from laminated cardboard or from any other convenient material sufficiently strong to maintain its shape so that the device can be reusable and can be disinfected between uses with, for example, an alcohol wipe or towelette. Alternatively, the sizing card 70 can be disposable, meaning that the sizing card 70 is intended to be used for only one blood draw procedure and patient and then discarded. In such cases, the disposable sizing card 70 can be made from less substantial materials, such as thin cardboard or paper. The openings 72 can be provided on the sizing card 70 in any convenient orientation. For example, the openings 72 can be arranged as a vertical line (as shown in FIG. 2), in a horizontal line, a square (e.g., a 2×2 array of openings 72), or any other variety of arrangement.

In some examples, the openings are elliptical openings 72 having a major diameter D1 that corresponds to the maximum finger width that can be used for the device 10a, 10b, 10c, 10d. The openings 72 can also include a minor diameter D2 corresponding to a maximum acceptable finger height for each device 10a, 10b, 10c, 10d. The openings 72 can further include a notch 74 on the top of each opening 72. The purpose of the notch 74 is to make it easier for the patient to insert his or her finger into and/or remove his or her finger from the opening 72 during sizing.

In some examples, the major diameter D1 and the minor diameter D2 of the top opening 72 correspond to maximum acceptable dimensions for the small sized blood collection device 10a. The bottom opening 72 can have a major diameter D1 and a minor diameter D2 corresponding to maximum acceptable dimensions of the extra-large sized blood collection device 10d. In order to use the sizing card 70, the user assists the patient to insert his or her finger through the top opening 72 of the sizing card 70 to see if the small device 10a is appropriate for the patient. If the patient's finger passes through the top opening 72 up to or slightly past the first knuckle, then the small device 10a should be used for the particular patient. If the patient's finger does not pass through the top opening 72 up to or slightly past the first knuckle, then the patient's finger should be inserted through the opening 72 for the medium device 10b, the large device 10c, and the extra-large device 10d, in order to determine which opening 72 allows the patient's finger to pass through the opening 72 to or slightly past the first knuckle. In general, the user should use the size of blood collection device 10a, 10b, 10c, 10d corresponding to the smallest opening 72 that allows the patient's finger to pass through the opening 72 up to or slightly past the first knuckle.

With continued reference to FIG. 2, in some examples, the sizing card 70 may also include instructions 76 for using the sizing card 70 to determine correct sizing for the blood collection devices 10a, 10b, 10c, 10d. As shown in FIG. 2, the instructions 76 are printed directly on the card shown to the user (i.e., the healthcare worker and/or patient) that the patient's finger should pass through the opening 72 up to or slightly beyond the first knuckle. Providing clear and easily accessible instructions is believed to reduce a possibility of user error and improve compliance (i.e., that the user and/or patient will use the device size indicated by the sizing card 70).

In some examples, the blood collection devices 10a, 10b, 10c, 10d of the set 62 are marked with numbers or other indicators to help the user to identify the correct device for a particular blood draw procedure. For example, the numbers or indicators can be molded on the device 10, as shown in FIG. 1C. In some examples, the small device 10a can be designated number “1”, the medium device 10b can be designated number “2”, the large device 10c can be designated number “3”, and the extra-large device can be designed number “4”. As shown in FIG. 2, the sizing card 70 includes the numbers printed in close proximity to the openings 72 so that the user knows which opening 72 corresponds to which device 10a, 10b, 10c, 10d. In other examples, the devices 10a, 10b, 10c, 10d can be identified by letters (i.e., S, M, L, and EX) or other visual indicators or drawings to help users determine which device 10a, 10b, 10c, 10d of the set or kit 62 should be used for a particular patient's finger.

In some examples, colors may also be used to indicate the size of each device 10a, 10b, 10c, 10d. For example, the small device 10a can be coated and/or molded from a material that is pink in color; the medium device 10b can be coated with and/or molded from an orange material; the large device 10c can be coated with and/or molded from a green material; and the extra-large device 10d can be coated with and/or molded from a blue material. In such cases, in order to improve user compliance, the sizing card 70 can be designed with colors corresponding to colors of the blood collection devices 10a, 10b, 10c, 10d. For example, areas 78 of the sizing card 70 surrounding each opening 72 can be a color corresponding to the color of the device 10a, 10b, 10c, 10d. Specifically, there can be a pink square or rectangle surrounding the top opening 72, which corresponds to the small device 10a. Similarly, there can be an orange square or rectangle surrounding the opening 72 for the medium device; a green square or rectangle surrounding the large opening 72; and a blue square or rectangle surrounding the extra-large opening 72.

In some examples, the colors (i.e., pink, orange, green, and blue) printed on the sizing card 70 and/or a hue or shade of each color can be selected to allow persons of the population who are color blind to use the sizing card 70. In particular, colors and/or shades of materials or coatings of the devices 10a, 10b, 10c, 10d and colors and/or shades printed on the sizing card 70 can be selected so that individuals with deuteranopia (green color blindness), protanopia (red color blindness), tritanopia (blue color blindness), and/or monochromacy (complete color blindness) are able to distinguish between the different colors and/or shades. It is believed that, in many cases, colorblind individuals can distinguish between the different colors printed on the sizing card 70 shown in FIG. 2 (i.e., pink, orange, green, and blue). However, while the combination of colors in FIG. 2 is believed to be appropriate for many users, it is understood that other combinations of colors can be used and/or will be identified by those skilled in the art for particular users within the scope of the present disclosure.

While different examples of the set or kit of blood collection devices and associated sizing systems and methods are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

The invention claimed is:

1. A kit of parts for obtaining a capillary blood sample, the kit comprising:
a plurality of capillary blood collection devices of different sizes; and
a sizing tool for identifying which of the plurality of blood collection devices to use for a particular patient's finger,
wherein each blood collection device of the plurality of blood collection devices comprises a finger holder comprising a finger receiving portion designed for a unique ideal finger size and configured for use with fingers within a unique size range, and
wherein the unique size ranges for the plurality of blood collection device are selected such that a majority of fingers for a population of patients are within one of the unique size ranges for the plurality of capillary blood collection devices.

2. The kit of claim 1, wherein the ideal finger sizes comprise an ideal finger width, ideal finger height, and/or ideal finger length, and wherein the unique size ranges comprise a range of finger widths, a range of finger heights, and/or a range of finger lengths.

3. The kit of claim 1, wherein the finger holders of the blood collection devices further comprise an actuation portion and a port.

4. The kit of claim 3, wherein the blood collection devices each further comprise:
a container engagement portion connected to the holder; and
a collection container removably connectable to the container engagement portion, the container defining a collection cavity,
wherein the actuation portion comprises at least two wings configured to create pressure for the patient's finger positioned within the finger receiving portion.

5. The kit of claim 1, wherein at least 95% of the patient fingers for the population of patients are within one of the unique size ranges.

6. The kit of claim 1, wherein the population of patients comprises a population of all adult patients living within a selected geographic region, and wherein the finger sizes for the population of patients substantially matches a normal distribution.

7. The kit of claim 1, wherein a difference between a maximum value and a minimum value for each of the unique size ranges are equal to each other.

8. The kit of claim 7, wherein the unique ideal finger sizes for the plurality of blood collection devices are equally between the minimum value and the maximum value for each of the unique size ranges.

9. The kit of claim 1, wherein the unique size ranges for the plurality of blood collection devices are selected such that an equal number of patient fingers of the patient population are within the unique size range for each of the blood collection devices of the plurality of blood collection devices.

10. The kit of claim 1, wherein the unique ideal finger sizes for the plurality of blood collection devices are a minimum value of the unique size range for each of the plurality of blood collection devices.

11. The kit of claim 1, wherein the unique ideal finger sizes for the plurality of blood collection devices is a maximum value of the unique size range for each of the plurality of blood collection devices.

12. The kit of claim 1, wherein the unique ideal finger sizes for the plurality of blood collection devices is greater than a middle value of the unique size range for each of the plurality of blood collection devices.

13. The kit of claim 1, wherein the unique ideal finger sizes for the plurality of blood collection devices are selected such that a ratio of loose/tight fit for finger sizes within the unique size ranges are the same for each of the plurality of blood collection devices.

14. The kit of claim 1, wherein the kit comprises at least four blood collection devices of different sizes.

15. The kit of claim 1, wherein the sizing tool comprises a sizing card for a size exclusion determination, the sizing card comprising a plurality of elliptical openings, wherein each of the plurality of elliptical openings is sized to correspond to a maximum value of the unique size range for one of the plurality of blood collection devices.

16. The kit of claim 15, wherein the plurality of elliptical openings each comprise a major diameter corresponding to a maximum finger width and a minor diameter corresponding to a maximum finger height for the unique size range for the one of the plurality of blood collection devices.

17. The kit of claim 1, wherein the sizing tool comprises at least one measuring tool, such as a ruler or calipers, configured to directly measure at least one of a width, height, and/or length of the patient's finger.

18. The kit of claim 1, wherein the sizing tool comprises a computer processor configured to:
receive at least one image of the patient's finger;
process the received at least one image to determine at least one of a finger width, height, and/or length of the patient's finger; and
cause an output device to display an indication of which of the plurality of blood collection devices to use for the patient's finger determined based, at least in part, on the finger width, height, and/or length of the patient's finger determined by the processing of the at least one image and the unique size ranges for the plurality of blood collection devices.

19. A method of making a plurality of blood collection devices sized for use with at least a majority of patient finger sizes for a patient population, the method comprising:
determining unique size ranges for each of the plurality of blood collection devices;
determining a unique ideal size for each device of the plurality of blood collection devices, wherein the unique ideal size for each device is within the determined size range for the respective device of the plurality of blood collection devices; and
making the plurality of blood collection devices with dimensions corresponding to the determined unique ideal size for each of the plurality of blood collection devices.

20. A computer-implemented method for determining a correct blood collection device size for a patient's finger, the method comprising:
receiving, with at least one computer processor, at least one image of the patient's finger;
processing, with the at least one computer processor, the received at least one image to determine at least one dimension of the patient's finger; and
causing, with the at least one computer processor, an output device to provide a visual indication of which blood collection device of a plurality of blood collection devices having unique finger size ranges to use for the patient's finger determined based, at least in part, on the determined at least one dimension of the patient's finger and the unique size ranges for the plurality of blood collection devices.
